(19) United States
(12) Patent Application Publication
(10) Pub. No.: US 2013/0303816 A1
(43) Pub. Date: Nov. 14, 2013

(54) Catalysts with Carbonaceous Material for Improved CUMENE Production and Method of Making and Using Same

(71) Applicants: Deng-Yang Jan, Elk Grove Village, IL (US); Jacob M. Anderson, Downers Grove, IL (US); Pelin Cox, Shaumburg, IL (US)

(72) Inventors: Deng-Yang Jan, Elk Grove Village, IL (US); Jacob M. Anderson, Downers Grove, IL (US); Pelin Cox, Shaumburg, IL (US)

(73) Assignee: UOP LLC, Des Plaines, IL (US)

(21) Appl. No.: 13/471,118

(22) Filed: May 14, 2012

(51) Int. Cl. C07C 2/66; B01J 37/02; B01J 29/06

(52) U.S. Cl. 585/467; 502/60; 502/64; 502/62

(57) ABSTRACT

A composite catalyst is presented. The composite catalyst comprises a substrate. The substrate comprises a zeolite and an inorganic oxide. The composite further comprises a carbonaceous material disposed on a surface of the substrate. The carbonaceous material comprises greater than about 2.8 weight percent of the composite catalyst.

### FIELD OF THE INVENTION

The disclosure relates in general to the formation of isopropylbenzene (cumene) through catalytic alkylation of benzene. In certain embodiments, the disclosure relates forming a carbonaceous material on the surface of a catalyst to increase cumene selectivity.

### BACKGROUND OF THE INVENTION

Zeolites are crystalline aluminosilicate compositions which are microporous and which are formed from corner sharing AlO₂ and SiO₂ tetrahedra. Numerous zeolites, both naturally occurring and synthetically prepared are used in various industrial processes. Synthetic zeolites are prepared via hydrothermal synthesis employing suitable sources of Si, Al, as well as structure directing agents such as alkali metals, alkaline earth metals, amines, or organoammonium cations. The structure directing agents reside in the pores of the zeolite and are largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase which is dispersed throughout the internal voids of the crystal without significantly displacing any atoms which make up the permanent zeolite crystal structure. Zeolites can be used as catalysts for hydrocarbon conversions, which can take place on outside surfaces as well as on internal surfaces within the pore.

One such hydrocarbon conversion process includes the catalytic monoalkylation of benzene with propylene to produce isopropylbenzene (cumene) using a zeolitic catalysts. While the primary product is isopropylbenzene, quantities of polyalkylated benzene variants are also produced in small quantities. These polyalkylated variants, such as diisopropylbenzene (DIPB) and triisopropylbenzene (TIPB), are undesirable. As such, technology to increase the selectivity of the catalytic alkylation to isopropylbenzene over the polyalkylated variants is very much desired. Those skilled in the art recognize the significant commercial impact of even a modest improvement in product selectivity.

### SUMMARY OF THE INVENTION

A composite catalyst is presented. The composite catalyst comprises a substrate. The substrate comprises a zeolite and an inorganic oxide. The composite catalyst further comprises a carbonaceous material disposed on a surface of the substrate. The carbonaceous material comprises greater than about 2.8 weight percent of the composite.

In another embodiment, a method of making a composite catalyst is presented. The method comprises providing a substrate comprising a zeolite and an inorganic oxide and depositing a carbonaceous material on a surface of the substrate. The depositing comprises exposing the substrate to a hydrocarbon material in the vapor phase or partial vapor phase. The carbonaceous material comprises greater than about 2.8 weight percent of the composite catalyst.

In yet another embodiment, a method of making cumene is presented. The method comprises providing a substrate comprising a zeolite and an inorganic oxide and depositing a carbonaceous material on a surface of the substrate. The depositing comprises exposing the substrate to a hydrocarbon material in the vapor phase or partial vapor phase. The carbonaceous material comprises greater than about 2.8 weight percent of the composite catalyst. The method further comprises forming cumene by exposing the composite catalyst to a stream comprising propylene and benzene. The stream comprises a liquid.

### BRIEF DESCRIPTION OF THE DRAWINGS

FIG. 1 is a graph of carbon content versus cumene selectivity;

FIG. 2 is a graph of carbon content versus activity (the portion of the catalyst bed required to attain maximum temperature); and

FIG. 3 is a graph of carbon content versus percent bridgehead carbon.

### DETAILED DESCRIPTION OF PREFERRED EMBODIMENTS

The catalytic compositions which are used in the processes of the current invention comprise cumene alkylation catalyst UZM-8. UZM-8 is a microporous crystalline zeolite. In one embodiment, UZM-8 is prepared in an alkali-free reaction medium in which only one or more organoammonium species are used as structure directing agents. The UZM-8 zeolite has a composition in the as-synthesized form and on an anhydrous basis expressed by empirical formula (1).

Rᵣ^{p+Al}₁₋ₓEₓSi_{y}O_{z} (1)

In various embodiments, R is at least one organoammonium cation selected from the group consisting of protonated amines, protonated diamines, quaternary ammonium ions, diquaternary ammonium ions, protonated alkanolamines and quaternized alkanolammonium ions.

In one embodiment, the organoammonium cations are non-cyclic. In one embodiment, the organoammonium cations do not comprise a cyclic group as one substituent. In one embodiment, the organoammonium cations comprise at least one methyl group as a substitute. In one embodiment, the organoammonium cations comprise at least two methyl groups as substituents. In certain embodiments, the cations are selected from the group consisting of diethyldimethylammonium (DEDMA), ethyltrimethylammonium (ETMA), hexamethonium (HM) and mixtures thereof.

The ratio of R to (Al+E) is represented by “r” which varies from about 0.05 to about 5. The value of “p” which is the weighted average valence of R varies from about 1 to about 2. The ratio of Si to (Al+E) is represented by “y” which varies from about 6.5 to about 35. E is an element which is tetrahedrally coordinated and present in the framework. In certain embodiments, E is selected from the group consisting of gallium, iron, chromium, indium and boron. The mole fraction of E is represented by “x” and has a value from about 0 to about 0.5, while “z” is the mole ratio of 0 to (Al+E) and is given by equation (2).

z=(r·p+3+4·y)/2 (2)

The UZM-8 zeolites can also be prepared using both organoammonium cations and alkali and/or alkaline earth cations as structure directing agents. As in the alkali-free case above, the same organoammonium cations can be used here. Alkali or alkaline earth cations are observed to speed up the crystallization of UZM-8, often when present in amounts less than 0.05 M⁺/Si. For the alkali and/or alkaline earth metal containing systems, the microporous crystalline UZM-8 zeolite has a composition in the as-synthesized form and on an anhydrous basis expressed by empirical formula (3).

Mₘⁿ⁺Rᵣ^{p+}Al₁₋ₓEₓSi_{y}O_{z} (3)

M is at least one exchangeable cation. In certain embodiments, M is selected from the group consisting of alkali and alkaline earth metals. In various embodiments, M comprises lithium, sodium, potassium, rubidium, cesium, calcium, strontium, barium, or mixtures thereof. In certain embodiments, R is selected from the group consisting of DEDMA, ETMA, HM, and mixtures thereof.
The value of “m” which is the ratio of M to (Al+E) varies from about 0.01 to about 2. The value of “n” which is the weighted average valence of M varies from about 1 to about 2. The ratio of R to (Al+E) is represented by “r” which varies from 0.05 to about 5. The value of “p” which is the weighted average valence of R varies from about 1 to about 2. The ratio of Si to (Al+E) is represented by “y” which varies from about 6.5 to about 35. E is an element which is tetrahedrally coordinated and present in the framework. In certain embodiments, E is selected from the group consisting of gallium, iron, chromium, indium and boron.
The mole fraction of E is represented by “x” and has a value from about 0 to about 0.5, while “z” is the mole ratio of 0 to (Al+E) and is given by equation (4).

z=(m·n+r·p+3+4·y)/2 (4)

In some embodiments, where M consists of a single metal, the weighted average valence is the valence of the metal, i.e. +1 or +2. In other embodiments, where M consists of a plurality of metals, the total metal amount is represented by (5) and the weighted average valence “n” is given by equation (6).

$$\begin{matrix}{M}_{m}^{n +} = {M}_{m 1}^{( n 1 ) +} + {M}_{m 2}^{( n 2 ) +} + {M}_{m 3}^{( n 3 ) +} + … & ( 5 ) \\ n = \frac{{m}_{1} \cdot {n}_{1} + {m}_{2} \cdot {n}_{2} + {m}_{3} \cdot {n}_{3} + …}{{m}_{1} + {m}_{2} + {m}_{3} …} & ( 6 )\end{matrix}$$

Similarly when only one R organic cation is present, the weighted average valence is the valence of the single R cation, i.e., +1 or +2. When more than one R cation is present, the total amount of R is given by equation (7).

Rᵣ^{p+}=Rᵣ₁^{(p1)+}+Rᵣ₂^{(p2)+}+Rᵣ₃^{(p3)+} (7)

and the weighted average valence “p” is given by the equation (8).

$$p = \frac{{p}_{1} \cdot {r}_{1} + {p}_{2} \cdot {r}_{2} + {p}_{3} \cdot {r}_{3} + …}{{r}_{1} + {r}_{2} + {r}_{3} …} \qquad ( 8 )$$

In various embodiments, the microporous crystalline UZM-8 zeolites are prepared by a hydrothermal crystallization of a reaction mixture prepared by combining reactive sources of R, aluminum, and silicon. In various embodiments, the microporous crystalline UZM-8 zeolites are prepared by a hydrothermal crystallization of a reaction mixture prepared by combining reactive sources of R, aluminum, silicon, and M. In various embodiments, the microporous crystalline UZM-8 zeolites are prepared by a hydrothermal crystallization of a reaction mixture prepared by combining reactive sources of R, aluminum, silicon, and E. In various embodiments, the microporous crystalline UZM-8 zeolites are prepared by a hydrothermal crystallization of a reaction mixture prepared by combining reactive sources of R, aluminum, silicon, M and E.

In various embodiments, the UZM-8 zeolites comprise a three dimensional lattice with recessed cups formed on the surface of the catalyst and pores extending through the lattice.

In various embodiments, the source of aluminum is selected from the group consisting of aluminum alkoxides, precipitated aluminas, aluminum metal, sodium aluminate, organoammonium aluminates, aluminum salts, and alumina sols. Specific examples of aluminum alkoxides include, but are not limited to aluminum ortho sec-butoxide, and aluminum ortho isopropoxide. Other sources of aluminum may be used in other embodiments.

In various embodiments, the source of silica is selected from the group consisting of tetraethylorthosilicate, colloidal silica, precipitated silica, alkali silicates, and organoammonium silicates. Other sources of silica may be used in other embodiments.

A special reagent consisting of an organoammonium aluminosilicate solution can also serve as the simultaneous source of Al, Si, and R.

In various embodiments, the source of E is selected from the group consisting of alkali borates, boric acid, precipitated gallium oxyhydroxide, gallium sulfate, ferric sulfate, ferric chloride, chromium nitrate, and indium chloride. Other sources of E may be used in other embodiments.

In various embodiments, the source of M is selected from the group consisting of halide salts, nitrate salts, sulfate salts, acetate salts, and hydroxides of the respective alkali or alkaline earth metals. Other sources of M may be used in other embodiments.

In certain embodiments, R can be introduced as an organoammonium cation or an amine. In the embodiments where R is a quaternary ammonium cation or a quaternized alkanolammonium cation, the source of R may be selected from the group consisting of hydroxide, chloride, bromide, iodide and fluoride compounds. Specific examples of such sources of R include without limitation DEDMA hydroxide, ETMA hydroxide, tetramethylammonium hydroxide, tetraethylammonium hydroxide, hexamethonium hydroxide, tetrapropylammonium hydroxide, methyltriethylammonium hydroxide, tetramethylammonium chloride, and choline chloride. In some embodiments, R may be introduced as an amine, diamine, or alkanolamine that subsequently hydrolyzes to form an organoammonium cation. In some embodiments, the source of R is selected from the group consisting of N,N,N,N-tetramethyl-1,6-hexanediamine, triethylamine, and triethanolamine. In some embodiments, the source of R is selected from the group consisting of ETMAOH, DEDMAOH, and HM(OH)2.

The reaction mixture containing reactive sources of the desired components can be described in terms of molar ratios of the oxides by formula (9).

aM_{2/n}O:bR_{2/}pO:1-cAl₂O3:cE₂O₃:dSiO₂:eH₂O (9)

In various embodiments, “a” varies from 0 to about 25, “b” varies from about 1.5 to about 80, “c” varies from 0 to 1.0, “d” varies from about 10 to about 100, and “e” varies from about 100 to about 15000. If alkoxides are used, it is preferred to include a distillation or evaporative step to remove the alcohol hydrolysis products.

The reaction mixture is reacted at a temperature of about 85° C. to about 225° C. and preferably from about 125° C. to about 150° C. for a period of about 1 day to about 28 days and preferably for a time of about 5 days to about 14 days in a sealed reaction vessel under autogenous pressure. After crystallization is complete, the solid product is isolated from the heterogeneous mixture by means such as filtration or centrifugation, and then washed with deionized water and dried in air at ambient temperature up to about 100° C.

In some embodiments, the UZM-8 is synthesized from a homogenous solution. Soluble aluminosilicate precursors condense during digestion to form extremely small crystallites that have a great deal of external surface area and short diffusion paths within the pores of the crystallites. This can affect both adsorption and catalytic properties of the material.

As-synthesized, the UZM-8 material will contain some of the charge balancing cations in its pores. In the case of syntheses from alkali or alkaline earth metal-containing reaction mixtures, some of these cations may be exchangeable cations that can be exchanged for other cations. In the case of organoammonium cations, they can be removed by heating under controlled conditions. In the cases where UZM-8 is prepared in an alkali-free system, the organoammonium cations are best removed by controlled calcination, thus generating the acid form of the zeolite without any intervening ion-exchange steps. On the other hand, it may sometimes be possible to remove a portion of the organoammonium via ion exchange. In a special case of ion exchange, the ammonium form of UZM-8 may be generated via calcination of the organoammonium form of UZM-8 in an ammonia atmosphere.

The properties of the UZM-8 compositions described above can be modified by removing some of the aluminum atoms from the framework and optionally inserting silicon atoms. Treating processes include, without limitation, treatment with a fluorosilicate solution or slurry, extraction with a weak, strong or complexing acid, etc. In carrying out these dealumination treatments, the particular form of the UZM-8 is not critical, but can have a bearing on the final product especially with regard to the extent of dealumination.

Thus, the UZM-8 can be used as synthesized or can be ion exchanged to provide a different cation form. In this respect, the starting zeolite can be described by empirical formula (10).

M′_{m′}^{n′+}R_{r′}^{p+}Al₍₁₋ₓ₎EₓSi_{y}O_{z′} (10)

R, x, y, and E are as described above and m′ has a value from about 0 to about 7.0, M′ is a cation selected from the group consisting of alkali metals, alkaline earth metals, rare earth metals, hydrogen ion, ammonium ion, and mixtures thereof, n′ is the weighted average valence of M′ and varies from about 1 to about 3, r′ has a value from about 0 to about 7.0, r′+m′>0, and p is the weighted average valence of R and varies from about +1 to about +2. The value of z′ is given by the formula (II).

z′=(m′·n′+r′·p+3+4·y)/2 (11)

The UZM-8 catalyst is used as a catalyst or a catalyst support for a number of hydrocarbon conversion processes known in the art. These include cracking, hydrocracking, alkylation of both aromatics and isoparaffins, isomerization, polymerization, reforming, dewaxing, hydrogenation, dehydrogenation, transalkylation, dealkylation, hydration, dehydration, hydrotreating, hydrodenitrogenation, hydrodesulfurization, methanation and syngas shift process.

In many hydrocarbon conversion processes, the zeolite is mixed with a binder for convenient formation of catalyst particles in a proportion of about 5 to 95 mass % zeolite and 5 to 95 mass % binder, with the zeolite, in some embodiments, comprising from about 10 to 90 mass % of the composite.

In various embodiments, the catalyst comprises between about 2 weight percent to about 80 weight percent zeolite. In various embodiments, the catalyst comprises between about 50 weight percent to about 70 weight percent zeolite. In various embodiments, the catalyst comprises between about 20 weight percent to about 98 weight percent inorganic oxide.

In various embodiments, the binder is porous, has a surface area of about 5 m²/g to about 800 m²/g, and is relatively refractory to the conditions utilized in the hydrocarbon conversion process. In various embodiments, the binders comprise an inorganic oxide. In various embodiments, the binders comprise, without limitation, alumina, titania, zirconia, zinc oxide, magnesia, boria, silica-alumina, silica-magnesia, chromia-alumina, alumina-boria, silica-zirconia, silica, silica gel, and clays. In various embodiments, the binders comprise amorphous silica and alumina, including gamma-, eta-, and theta-alumina.

In various embodiments, the zeolite with or without a binder are formed into various shapes such as pills, pellets, extrudates, spheres, etc. In some embodiments, the extrudates are prepared by conventional means, involves mixing the zeolite either before or after adding metallic components, with the binder and a suitable peptizing agent to form a homogeneous dough or thick paste having the correct moisture content to allow for the formation of extrudates with acceptable integrity to withstand direct calcination. The dough then is extruded through a die to give the shaped extrudate. A multitude of different extrudate shapes are possible, including, but not limited to, cylinders, cloverleaf, dumbbell and symmetrical and asymmetrical polylobes. In some embodiments, the extrudates are shaped to any desired form, such as spheres, by any means known to the art.

In some embodiments, the zeolite can be formed into a sphere by the oil-drop method described in U.S. Pat. No. 2,620,314, which is incorporated by reference. The method involves dropping a mixture of zeolite, and for example, alumina sol, and gelling agent into an oil bath maintained at elevated temperatures. The droplets of the mixture remain in the oil bath until they set and form hydrogel spheres. The spheres are then continuously withdrawn from the oil bath and typically subjected to specific aging treatments in oil and an ammoniacal solution to further improve their physical characteristics. The resulting aged and gelled particles are then washed and dried at a relatively low temperature of about 50-200° C. and subjected to a calcination procedure at a temperature of about 450-700° C. for a period of about 1 to about 20 hours. This treatment effects conversion of the hydrogel to the corresponding alumina matrix.

Generally speaking, the UZM-8 catalyst comprises a framework Si/Al₂ molar ratio in the range of about 19-22, whereas the UZM-8HR catalyst comprises a framework Si/Al₂ molar ratio in the range of about 23-35.

One of the uses of the formed UZM-8 zeolite catalyst (which includes both UZM-8 and UZM-8HR) is to catalyze alkylation and preferably the monoalkylation of aromatic compounds. In these applications, an aromatic compound is reacted with an olefin using the UZM-8 zeolitic catalyst. In various embodiments, the olefins comprises from 2 to about 20 carbon atoms. In various embodiments, the olefins comprise branched olefins or linear olefins and either terminal or internal olefins. In various embodiments, the olefins comprise ethylene, propylene, olefins which are known as “detergent range olefins,” or a combination thereof. “Detergent range olefins” are linear olefins containing from 6 up through about 20 carbon atoms which have either internal or terminal double bonds. In certain embodiments, the olefins comprise linear olefins containing from 8 to about 16 carbon atoms. In certain embodiments, the olefins comprise linear olefins containing from 10 to about 14 carbon atoms.

In various embodiments, the UZM-8 zeolitic catalyst is used to catalyze alkylation of benzene, naphthalene, anthracene, phenanthrene, and substituted derivatives thereof. In one embodiment, catalysts UZM-8 and UZM-8HR are used for catalytic, monoalkylation of benzene with propylene to produce isopropoylbenzene (cumene). While the primary product is isopropoylbenzene, quantities of polyalkylated benzene variants are also produced in small quantities. These polyalkylated variants, such as diisopropylbenzene (DIPB) and triisopropylbenzene (TIPB), are undesirable.

For example, monoalkylation of benzene is illustrated by (15), where benzene (12) is reacted with propylene (13) to form cumene (isopropylbenzene) (14).

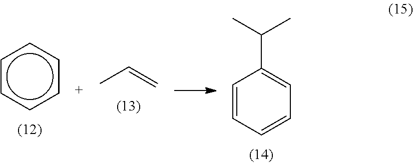

In other embodiments, the UZM-8 catalyst is used to catalyze alkylation of other aromatic compounds by an olefinic compound. In various embodiments, such aromatic compounds have one or more substituents selected from the group consisting of alkyl groups (having from 1 to about 20 carbon atoms), hydroxyl groups, and alkoxy groups whose alkyl group also contains from 1 up to 20 carbon atoms. In embodiments where the substituent is an alkyl or alkoxy group, a phenyl group can also can be substituted on the alkyl chain. In some embodiments, such aromatic compounds comprise biphenyl, toluene, xylene, ethylbenzene, propylbenzene, butylbenzene, pentylbenzene, hexylbenzene, heptylbenzene, octylbenzene, phenol, cresol, anisole, ethoxy-, propoxy-, butoxy-, pentoxy-, hexoxybenzene, and combinations thereof.

The particular conditions for the monoalkylation reaction depend upon the aromatic compound and the olefin used. In various embodiments, the reaction is conducted under at least partial liquid phase conditions. Therefore, the reaction pressure is adjusted to maintain the olefin at least partially dissolved in the liquid phase. For higher olefins, the reaction may be conducted at autogenous pressure. As a practical matter, the pressure normally is in the range between about 200 and about 1,000 psig (1480-6997 kPa) but usually is in a range between about 300-600 psig (2170-4238 kPa). The alkylation of aromatic compounds with the olefins in the C2-C20 range can be carried out at a temperature of about 60° C. to about 400° C., and in some embodiments, from about 90° C. to about 250° C., for a time sufficient to form the desired product. In some embodiments, the alkylation of benzene with ethylene is carried out at temperatures of about 200° C. to about 250° C. and the alkylation of benzene by propylene at a temperature of about 90° C. to about 200° C. The ratio of aromatic compound to olefin will depend upon the degree of selective monoalkylation desired as well as the relative costs of the aromatic and olefinic components of the reaction mixture. For alkylation of benzene by propylene, benzene-to-olefin ratios may be as low as about 1 and as high as about 10, with a ratio of 1.5-8 being preferred. Where benzene is alkylated with ethylene, a benzene-to-olefin ratio is, in one embodiment, between about 1:1 and 8:1. For detergent range olefins of C6-C20, a benzene-to-olefin ratio of between 2:1 up to as high as 30:1 is generally sufficient to ensure the desired monoalkylation selectivity, with a range between about 5:1 and about 20:1 even more preferred.

In various embodiments, the UZM-8 zeolitic catalyst is used to catalyze transalkylation. Transalkylation involves intermolecular transfer of the alkyl group on one aromatic nucleus to a second aromatic nucleus. In one embodiment, the transalkylation involves the transfer of one or more alkyl groups of a polyalkylated aromatic compound to a nonalkylated aromatic compound, and is exemplified by reaction of diisopropylbenzene (16) with benzene (17) to give two molecules of cumene (18) via reaction (19).

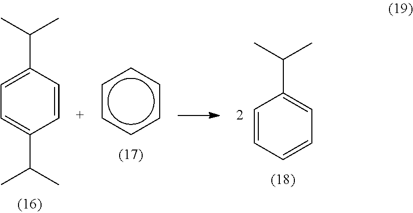

Transalkylation often is utilized to add to the selectivity of a desired selective monoalkylation by reacting the polyalkylates invariably formed during alkylation with nonalkylated aromatic to form additional monoalkylated products. For the purposes of this process, the polyalkylated aromatic compounds are those formed in the alkylation of aromatic compounds with olefins as described above, and the nonalkylated aromatic compounds are benzene, naphthalene, anthracene, and phenanthrene. The reaction conditions for transalkylation are similar to those for alkylation, with temperatures being in the range of about 100° C. to about 250° C., pressures in the range of about 100 to about 750 psig (about 791 kPa to about 5272 kPa), and the molar ratio of unalkylated aromatic to polyalkylated aromatic in the range from about 1 to about 10. Examples of polyalkylated aromatics that may be reacted with, for example, benzene as the nonalkylated aromatic, include without limitation, diethylbenzene, diisopropylbenzene, dibutylbenzene, triethylbenzene, triisopropylbenzene and tetraethylbenzene.

In processes where the UZM-8 catalyst is used to catalyze alkylation of benzene with propylene to produce cumene, the catalytic reaction generally occurs on the active sites that reside on the external surfaces of the catalyst. Applicant has observed that cumene selectivity can be enhanced by modifying the external surface of the catalyst to passivate surface functional groups. More specifically, this involves passivating the outer surface functional groups while leaving the framework acidic hydroxyl functional groups in the exposed cages of the catalyst relatively intact. As such, Applicant has developed a process for treating UZM-8-based zeolite catalysts, to increase cumene selectivity by passivating surface functional groups. When Applicant's catalyst is used in conventional cumene production processes involving benzene monoalkylation, Applicant's catalyst results in increased selectivity of cumene over the polyalkylated benzene variants.

In various embodiments, a carbonaceous material is disposed on the surface of the UZM-8 catalyst. As used herein, the term “substrate” refers to the catalyst before the addition of the carbonaceous material and the term “composite” and “composite catalyst” refers to the zeolitic catalyst. The carbonaceous material covers and therefore selectively passivates the surface active sites. This selective passivation results in an increased selectivity for isopropylbenzene. Without wishing to be bound by any particular theory, while the catalytic monoalkylation of benzene generally occurs in the exposed cages on the external surface of UZM-8 catalyst, it is believed that the acidic and non-acidic hydroxyl functional groups on the exterior surface of the catalyst (other than within the exposed cages) induce secondary alkylation reactions resulting in polyalkylated benzene variants. In short, it is believed that the surface active sites are responsible for the formation of polyalkylates due to the lack of geometric constraint imposed onto the active sites on the external surface of the catalyst. As such, the increase in selectivity observed using Applicants' catalyst is likely due to the carbonaceous material selectively passivating active acid sites on the surface of UZM-8 the catalyst, while leaving the active acid sites within the exposed cages of the catalyst unaffected.

Production of cumene from the catalytic monoalkylation of benzene generally takes place in the liquid phase stream of benzene and propylene at a temperature of between 60 and 260° C. Vapor phase conditions (i.e., low pressure and/or high temperature conditions) during cumene formation are avoided because such conditions lead to deactivation of the catalyst and low cumene selectivity. Applicants have found, however, that exposure to these extreme conditions for a period of time is useful in forming a carbonaceous material on the catalyst surface to increase cumene selectivity.

In one embodiment, the carbonaceous material is formed on the surface of the catalyst by treating the catalyst in a vapor phase or partial vapor phase hydrocarbon feed. In various embodiments, depending on the composition of the hydrocarbon feed, the pressure is between about 0.1 psia (0.7 kPa) and 550 psia (3792 kPa) and the temperature is between about 100° C. to about 450° C. In one embodiment, the process time is between about 0.02 and about 144 hours. In one embodiment, the process time is 24 hours.

In one embodiment, the hydrocarbon feed comprises an aromatic, an olefin, or a combination thereof. In one embodiment, the hydrocarbon feed comprises benzene and propylene in the same relative amounts as the feed for cumene production. In one embodiment, the hydrocarbon feed comprises benzene and an olefin at an olefin/benzene ratio of about 0.001 to about 300. In one embodiment, the hydrocarbon feed comprises benzene and an olefin at an olefin/benzene ratio of greater than about 1. In one embodiment, the hydrocarbon feed consists essentially of propylene. In one embodiment, the hydrocarbon feed consists essentially of benzene.

In one embodiment, the carbonaceous material is formed by the successive alkylation of a hydrocarbon to form a high molecular weight material. In one embodiment, the carbonaceous material is formed by the successive alkylation of benzene with oligomers, hydride transfer from long alkyl side chain to an olefin, cyclization and another hydride transfer to form a condensed aromatic ring. The formation of condensed aromatic rings is favored at high olefin to aromatic ratio at elevated temperatures in vapor phase. Furthermore, the increase in cumene selectivity is accompanied by an increase in the amount of carbonaceous material on the catalyst. The increased coke contents in turn are accompanied by an increase in the bridgehead carbon (as measured by C-13 NMR), which suggests the formation of condensed aromatic rings. In various embodiments, the hydrocarbon is propylene, benzene, or a combination thereof. As would be appreciated by those skilled in the art, any hydrocarbon, or combination of hydrocarbons, capable of alkylation and formation of condensed aromatic rings may be used to form the carbonaceous material. In various embodiments, the carbonaceous material comprises bridgehead carbon. In various embodiments, the carbonaceous material comprises greater than about 10 percent bridgehead carbon.

In one embodiment, the treated catalyst (i.e., having carbonaceous material disposed on its surface) is used in a traditional cumene production process to achieve higher levels of cumene selectivity as compared to a non-treated catalyst. In one embodiment, the treated catalyst is exposed to a liquid stream comprising benzene and propylene to achieve higher levels of cumene selectivity as compared to a non-treated catalyst.

In one embodiment, for each 1 weight percent of carbonaceous material added to the catalyst, the cumene selectivity increases by 3.1 percent. In one embodiment, for each 1 weight percent of carbonaceous material added to the catalyst, the cumene selectivity increases by 0.35 percent. In various embodiments, the carbonaceous material comprises greater than 1 weight percent of the catalyst. In various embodiments, the carbonaceous material comprises greater than 2.8 weight percent of the catalyst. In various embodiments, the carbonaceous material comprises between about 1 percent and 12 percent of the catalyst. In various embodiments, the carbonaceous material comprises between about 3.5 percent and 8 percent of the catalyst.

In some embodiments, the catalyst is flushed to carry away any carbonaceous material not secured to the catalyst. In some embodiments, the catalyst is flushed with a stream of nitrogen. In some embodiments, the catalyst is flushed with a stream of benzene.

In one embodiment, the treated catalyst has minimal activity loss as defined by an increase of less than 50 percent of the end of the active zone, i.e., the percentage of catalyst bed required to attain maximal temperatures.

The following Examples are presented to further illustrate to persons skilled in the art how to make and use Applicants' catalyst. These Examples are not intended as a limitation, however, upon the scope of Applicant's invention.

### Example 1

The following method is used to prepare a UZM-8 zeolite with a Si/Al₂ molar ratio of about 20. In a large beaker, 160.16 grams of diethyldimethylammonium hydroxide is added to 1006.69 grams de-ionized water, followed by 2.79 grams of 50 wt % NaOH solution. Next, 51.48 grams of liquid sodium aluminate is added slowly and stirred for 20 minutes. Then, 178.89 grams of SiO₂ (sold in commerce as Ultrasil) is slowly added to the gel and stirred for 20 minutes. Next, 24 grams of UZM-8 seed is added to the gel and stirred for an additional 20 minutes. The gel is then transferred to a 2-liter stirred reactor and heated to 160° C. in 2 hours and subsequently crystallized for 115 hours. After digestion, the material is filtered and washed with de-ionized water and dried at 100° C. XRD (X-Ray Diffraction) analysis of the resulting material shows pure UZM-8. The elemental analysis by inductively coupled plasma—atomic emission spectroscopy (ICP-AES) shows a, Si/Al₂ molar ratio of 20. A portion of the zeolite was calcined at 600° C., ammonium exchanged and then calcined at 550° C. to obtain a BET surface area of 462 m²/g, a total pore volume of 1.607 cc/g, and a micropore volume of 0.105 cc/g by N₂ adsorption isotherm. Surface area and pore volume are calculated using nitrogen partial pressure p/pₒ data points ranging from about 0.03 to about 0.30 using the BET (Brunauer-Emmett-Teller) model method using nitrogen adsorption technique as described in ASTM D4365-95, Standard Test Method for Determining Micropore Volume and Zeolite Area of a Catalyst, and in the article by S. Brunauer et al., J. Am. Chem. Soc., 60(2), 309-319 (1938).

### Example 2

The following method is used to prepare a UZM-8 zeolite with a Si/Al₂ molar ratio of about 25. In a large makeup tank, the following components are added, 8796 grams of de-ionized water, 6683 grams of diethyldimethylammonium hydroxide (20% solution), 693 grams of liquid sodium aluminate, 3422 grams of SiO₂ and 402 grams of UZM-8 seed with a Si/Al₂ molar ratio of about 20. The resulting gel was then pumped to the 5-gallon reactor, followed by rinsing the makeup tank with 1000 grams of de-ionized water and pumping the rinse to the 5-gallon reactor. The final gel was crystallized at 150° C. for 153 hours with an agitation at 506 rpm. After digestion, the material was isolated by centrifuge followed by hot de-ionized water wash. XRD data shows a pure UZM-8 material. The resulting zeolite showed a Si/Al₂ molar ratio of 25.4 by elemental analysis using ICP-AES. A portion of the zeolite was calcined at 600° C., ammonium exchanged and then calcined at 550° C. to obtain a BET surface area of 372 m²/g, a total pore volume of 0.50 cc/g, and a micropore volume of 0.122 cc/g by N₂ adsorption isotherm.

### Example 3

The following method is used to prepare a UZM-8 zeolite with a Si/Al₂ molar ratio of about 24. This method of Example 1 is followed with the exception of 120 instead of 153 hours of crystallization time and a variation in the work-up procedure. At the end of the synthesis, the product was isolated by first screening off particles greater than 40 mesh, room temperature water washing, centrifuging to remove mother liquor, drying at 100° C., hot water wash and then drying at 100° C. The product has a Si/Al₂ molar ratio of 24.4 by elemental analysis using ICP-AES. A portion of the zeolite was calcined at 600° C., ammonium exchanged and then calcined at 550° C. to obtain a BET surface area of 444 m²/g, a total pore volume of 0.91 cc/g, and a micropore volume of 0.13 cc/g by N₂ adsorption isotherm.

In a typical catalyst preparation, the zeolite is mixed with HNO₃ peptized Catapal B alumina with 70/30, 50/50 or 30/70 zeolite/Al₂O₃ proportion on a weight basis, and extruded into either a cylindrical or a trilobed shape. The extrudate is dried at 110° C. for 4 hours, calcined at 600° C. for 1 hour, exchanged with ammonium nitrate solution, washed by de-ionized water, dried at 120° C. and finally activated 550° C. in flowing air.

To test the catalyst performance 25 grams of catalyst was mixed with quartz sand to fill the interstitial voids to ensure proper flow distribution before loaded into a ⅞″ ID standard steel reactor. The catalyst was dried down with flowing benzene pretreated using 3A dryer at 200° C. for 12 hours. After the drydown, the recycle benzene was introduced followed by propylene. The benzene to propylene molar ratio for the test was targeted at 2.0, with a product effluent to combined fresh feed ratio of 7.4 on a weight basis, propylene weight hourly space velocity of 1.04 hr⁻¹, an inlet temperature of 115° C. and an outlet pressure of 500 psig (3549 kPa). The product effluent was monitored by on-line GC. The catalyst activity was measured by the percentage of catalyst bed required to reach maximal temperature, i.e., the less catalyst required to attain the maximal temperature, the higher the catalyst activity. The selectivity to cumene was calculated based on the moles of cumene out to the total moles of cumene, diisopropylbenzene and triisopropylbenzene. At the conclusion of the test, the propylene was first cut off while benzene feed continued through the reactor until the benzene purity at the reactor outlet reaches 99%. Thereafter, the benzene feed was discontinued and the N₂ was introduced to purge the benzene before unloading the catalyst. The spent catalyst was unloaded from the reactor and the amount of cabonaceous material was measured by method ASTM 5291.

### Example 4

A catalyst was prepared from a UZM-8 zeolite with a Si/Al₂ molar ratio of about 20 using 70/30 zeolite/Al₂O₃ formulation (i.e., 70% zeolite) with a cylindrical shape and an apparent bulk density of about 0.50 g/cc. A carbonaceous material was disposed on the catalyst by treating the catalyst to a feed of benzene in the vapor phase at a rate of 50 grams/hour at a temperature of 400° C. and at a pressure of 150 psig (1136 kPa) for 24 hours. The carbonaceous content of the treated catalyst was 8.5 weight percent with a cumene selectivity of 83.3 percent and with an activity, as percent of the active zone, of 63 percent.

### Example 5

Example 5 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 220° C. and at a pressure of 200 psig (1480 kPa) for 24 hours. The carbonaceous content of the treated catalyst was 2.0 weight percent with a cumene selectivity of 80.9 percent. In successive tests, the carbonaceous content of the treated catalyst was between 7.31 percent and 7.73 percent at the inlet of the treatment chamber.

### Example 6

Example 6 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of between 425° C. and 450° C. and at a pressure of between 100 psig (791 kPa) and 175 psig (1308 kPa) for 24 hours. The carbonaceous content of the treated catalyst was 11.0 weight percent with a cumene selectivity of 84.0 percent and with an activity, as percent of the active zone, of 63 percent.

### Example 7

Example 7 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 250 grams/hour at a temperature of 220° C. and at a pressure of 500 psig (3549 kPa) for 24 hours. The carbonaceous content of the treated catalyst at the inlet of the treatment chamber was 3.68 weight percent.

### Example 8

Example 8 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 250 grams/hour at a temperature of 220° C. and at a pressure of 200 psig (1379 kPa) for 24 hours. The carbonaceous content of the treated catalyst at the inlet of the treatment chamber was 2.67 weight percent.

### Example 9

Example 9 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 250 grams/hour at a temperature of 220° C. and at a pressure of 100 psig (791 kPa) for 24 hours. The carbonaceous content of the treated catalyst at the inlet of the treatment chamber was 2.13 weight percent.

### Example 10

Example 10 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 250 grams/hour at a temperature of 220° C. and at a pressure of 50 psig (446 kPa) for 24 hours. The carbonaceous content of the treated catalyst at the inlet of the treatment chamber was 2.27 weight percent.

### Example 11

Example 11 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 350°C. and at a pressure of 200 psig (1480 kPa) for 24 hours. The carbonaceous content of the treated catalyst at the inlet of the treatment chamber was 3.34 weight percent.

### Example 12

Example 12 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 450° C. and at a pressure of 200 psig (1480 kPa) for 24 hours. The carbonaceous content of the treated catalyst was 20 percent at the inlet of the treatment chamber, 18.4 percent at the midpoint of the treatment chamber, and 15 percent at the outlet of the treatment chamber.

### Example 13

Example 13 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 350° C. and at a pressure of 50 psig (446 kPa) for 24 hours. The carbonaceous content of the treated catalyst was 1.7 percent at the inlet of the treatment chamber, 1.95 percent at the midpoint of the treatment chamber, and 2.22 percent at the outlet of the treatment chamber.

### Example 14

Example 14 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 450° C. and at a pressure of 50 psig (446 kPa) for 24 hours. The carbonaceous content of the treated catalyst was 5.25 percent at the inlet of the treatment chamber, 8.12 percent at the midpoint of the treatment chamber, and 8.28 percent at the outlet of the treatment chamber.

### Example 15

Example 15 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 375° C. and at a pressure of 50 psig (446 kPa) for 24 hours. The carbonaceous content of the treated catalyst was 0.702 percent at the inlet of the treatment chamber, 1.4 percent at the midpoint of the treatment chamber, and 1.84 percent at the outlet of the treatment chamber.

### Example 16

Example 16 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 400° C. and at a pressure of 50 psig (446 kPa) for 24 hours. The carbonaceous content of the treated catalyst was 2.85 percent at the inlet of the treatment chamber, 3.8 percent at the midpoint of the treatment chamber, and 4.01 percent at the outlet of the treatment chamber.

### Example 17

Example 17 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 450° C. and at a pressure of 100 psig (791 kPa) for 24 hours. The carbonaceous content of the treated catalyst was 8.54 percent at the inlet of the treatment chamber, 11.6 percent at the midpoint of the treatment chamber, and 11.9 percent at the outlet of the treatment chamber.

### Example 18

Example 18 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 400° C. and at a pressure of 150 psig (1136 kPa) for 24 hours. In a first run, the carbonaceous content of the treated catalyst was 4.77 percent at the inlet of the treatment chamber, 7.57 percent at the midpoint of the treatment chamber, and 8.64 percent at the outlet of the treatment chamber. In successive tests, the carbonaceous content of the treated catalyst was between 8.46 percent and 9.14 percent at the midpoint of the treatment chamber.

### Example 19

Example 19 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 350° C. and at a pressure of 100 psig (791 kPa) for 24 hours. The carbonaceous content of the treated catalyst was 1.07 percent at the inlet of the treatment chamber, 1.89 percent at the midpoint of the treatment chamber, and 2.34 percent at the outlet of the treatment chamber.

### Example 20

Example 20 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 425°C. and at a pressure of 175 psig (1308 kPa) for 24 hours. The carbonaceous content of the treated catalyst was 10.5 percent at the midpoint of the treatment chamber.

### Example 21

Example 21 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 450° C. and at a pressure of 150 psig (1136 kPa) for 24 hours. The carbonaceous content of the treated catalyst was 14.5 percent at the midpoint of the treatment chamber.

### Example 22

Example 22 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of benzene in the vapor phase at 50 grams/hour at a temperature of 400° C. and at a pressure of 200 psig (1480 kPa) for 24 hours. The carbonaceous content of the treated catalyst was 9.67 percent at the midpoint of the treatment chamber.

### Example 23

Example 23 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of propylene in the vapor phase at 25.7 grams/hour at a temperature of 200° C. and at a pressure of 50 psig (446 kPa) for 12+25 hours. The carbonaceous content of the treated catalyst was 31.6 percent at the inlet of the test chamber.

### Example 24

Example 24 was prepared from a UZM-8 zeolite catalyst having a cylindrical shape, a Si/Al₂ molar ratio of 20, an average bulk density of 0.5 g/cc, and comprising 70% zeolite. A carbonaceous material was disposed on the catalyst by treating the catalyst to a feed of propylene in the vapor phase at 25.7 grams/hour at a temperature of 270° C. and at a pressure of 50 psig (446 kPa) for 24 hours. The carbonaceous content of the treated catalyst was 32 percent at the inlet of the test chamber, 38.3 percent at the midpoint of the test chamber, and 35.8 percent at the outlet of the test chamber.

### Example 25

Example 25 was prepared from an UZM-8HR zeolite catalyst having a trilobe shape, a Si/Al₂ molar ratio of 25.4, an average bulk density of 0.627 g/cc, and comprising 50% zeolite. A carbonaceous material was disposed on the catalyst by treating the catalyst to a feed of propylene/benzene (with a ratio of 300 moles olefin per mole benzene) in the vapor phase at 25.7 grams/hour at a temperature of 267° C. and at a pressure of 500 psig (3549 kPa) for 24 hours. The carbonaceous content of the treated catalyst was 4.6 percent with a cumene selectivity of 87.4 percent and an activity, as a percent of the active zone, of 40 percent.

An analysis of the carbon using Carbon-13 NMR analysis showed that 21 to 29 percent of the carbonaceous material was bridgehead carbons. Overall, 80 to 83 percent of the carbonaceous material was aromatic and 20 to 17 percent was aliphatic.

### Example 26

Example 26 was prepared from an UZM-8HR zeolite catalyst having a trilobe shape, a Si/Al₂ molar ratio of >20, an average bulk density of 0.559 g/cc, and comprising 50% zeolite. A carbonaceous material was disposed on the catalyst by treating the catalyst to a feed of propylene/benzene (with a ratio of 2 moles olefin per mole benzene) in the vapor phase at 25.7 grams/hour at a temperature of 270° C. and at a pressure of 500 psig (3549 kPa) for 24 hours. The carbonaceous content of the treated catalyst was 2.7 percent with a cumene selectivity of 81.4 percent and an activity, as a percent of the active zone, of 45.4 percent.

### Example 27

Example 27 was prepared from an UZM-8HR zeolite catalyst having a trilobe shape, a Si/Al₂ molar ratio of 24.4, an average bulk density of 0.488 g/cc, and comprising 50% zeolite. A carbonaceous material was disposed on the catalyst by treating the catalyst to a feed of propylene/benzene (with a ratio of 1.08 moles olefin per mole benzene) in the vapor phase at 25.7 grams/hour at a temperature of 255° C. and at a pressure of 500 psig (3549 kPa) for 24 hours. The carbonaceous content of the treated catalyst was 3.8 percent with a cumene selectivity of 85.5 percent and an activity, as a percent of the active zone, of 42.1 percent.

An analysis of the carbon using Carbon-13 NMR analysis showed that 13 percent of the carbonaneous material was bridgehead carbons. Overall, 75 percent of the carbonaceous material was aromatic and 25 percent was aliphatic.

### Example 28

Example 28 was prepared from a UZM-8 zeolite catalyst having a trilobe shape, a Si/Al₂ molar ratio of 20, an average bulk density of 0.562 g/cc, and comprising 70% zeolite. A carbonaceous material was disposed on the catalyst by treating the catalyst to a feed of propylene/benzene (with a ratio of 0.9 moles olefin per mole benzene) in the vapor phase at 25.7 grams/hour at a temperature of 182° C. and at a pressure of 500 psig (3549 kPa) for 24 hours. The carbonaceous content of the treated catalyst was 2.5 percent with a cumene selectivity of 78.6 percent and an activity, as a percent of the active zone, of 35.7 percent.

### Example 29

Example 29 was prepared from a UZM-8 zeolite catalyst having a cylinder shape, a Si/Al₂ molar ratio of 20, an average bulk density of 0.562 g/cc, and comprising 70% zeolite. A carbonaceous material was disposed on the catalyst by treating the catalyst to a feed of propylene/benzene (with a ratio of 1.7 moles olefin per mole benzene) in the vapor phase at 25.7 grams/hour at a temperature of 268° C. and at a pressure of 500 psig (3549 kPa) for 24 hours. The carbonaceous content of the treated catalyst was 8.1 percent with a cumene selectivity of 88 percent and an activity, as a percent of the active zone, of 46.7 percent.

An analysis of the carbon using Carbon-13 NMR analysis showed that 24 to 28 percent of the carbonaceous material was bridgehead carbons (carbon atoms that connect different rings in the same molecule or that bride across an aromatic ring). Overall, 71 to 72 percent of the carbonaceous material was aromatic and 29 to 28 percent was aliphatic.

### Example 30

Example 30 was prepared from a UZM-8 zeolite catalyst having a trilobe shape, a Si/Al₂ molar ratio of 20, an average bulk density of 0.449 g/cc, and comprising 50% zeolite. A carbonaceous material was disposed on the catalyst by treating the catalyst to a feed of propylene/benzene (with a ratio of 0.5 moles olefin per mole benzene) in the vapor phase at 25.7 grams/hour at a temperature of 303° C. and at a pressure of 500 psig (3549 kPa) for 24 hours. The carbonaceous content of the treated catalyst was 4.9 percent with a cumene selectivity of 85.9 percent and an activity, as a percent of the active zone, of 50.3 percent.

### Example 31

Example 31 was prepared from a UZM-8 zeolite catalyst having a trilobe shape, a Si/Al₂ molar ratio of 20, an average bulk density of 0.496 g/cc, and comprising 70% zeolite. A carbonaceous material was disposed on the catalyst by treating the catalyst to a feed of propylene/benzene (with a ratio of 0.5 moles olefin per mole benzene) in the vapor phase at 25.7 grams/hour at a temperature of 307° C. and at a pressure of 500 psig (3549 kPa) for 24 hours. The cumene selectivity of the catalyst was 85.7 percent with an activity, as a percent of the active zone, of 38.6 percent.

### Example 32

Example 32 was prepared from a UZM-8 zeolite catalyst having a trilobe shape, a Si/Al₂ molar ratio of 20, an average bulk density of 0.496 g/cc, and comprising 70% zeolite. A carbonaceous material was disposed on the catalyst by treating the catalyst to a feed of propylene/benzene (with a ratio of 2.0 moles olefin per mole benzene) in the vapor phase at 25.7 grams/hour at a temperature of 141° C. and at a pressure of 500 psig (3549 kPa) for 24 hours. The cumene selectivity of the catalyst was 80.9 percent with an activity, as a percent of the active zone, of 36 percent.

The performance of a number of UZM-8-based catalysts with and without a carbonaceous material deposited by Applicants' method is provided in Table 1 below.

TABLE 1
Performance Without Carbonaceous Treatment

| | | | | | | | |
|---|---|---|---|---|---|---|---|
| Cumene Selectivity | 83.2 | 78.7 | 81.4 | 84.0 | 80.9 | 83.9 | 81.7 |
| activity, as % of active zone | 34.7 | 35.9 | 28.7 | 33.1 | 47.7 | 36.1 | 55.5 |
| TIPB/(cumene + DIPB + TIPB) | 1.0 | 1.8 | 1.2 | 0.8 | 1.8 | 0.9 | 1.3 |
| total feed benzene alkylated | 99.8 | 99.8 | 99.75 | 99.8 | 99.56 | 99.7 | 99.7 |
| DPE + DPP + PDPP sel, C-% | 0.06 | 0.08 | 0.1 | 0.06 | 0.05 | 0.07 | 0.09 |
| nPB/cumene | 100.0 | 86.0 | 93 | 97.0 | 85 | 101 | 105 |

Carbonaceous Treatment Conditions

| | | | | | | | |
|---|---|---|---|---|---|---|---|
| B/P min | 0.003 | 1.14 | 0.59 | 0.93 | 2 | 1.98 | 2.00 |
| hrs at min B/P | 0.083 | Minimal | 20 | Minimal | 22 | 20 | 25 |
| inlet temp | | 136 | 166 | 183 | 230 | 274 | 282 |
| max temp | 267 | 182 | 268 | 255 | 270 | 303 | 307 |

Performance After Carbonaceous Treatment

| | | | | | | | |
|---|---|---|---|---|---|---|---|
| Cumene Selectivity | 87.4 | 78.6 | 88.0 | 85.5 | 81.4 | 85.9 | 85.7 |
| activity, EAZ % | 40.0 | 35.7 | 46.7 | 42.1 | 45.4 | 50.3 | 38.57 |
| TIPB/(cumene + DIPB + TIPB) | 0.6 | 2.0 | 0.4 | 0.9 | 1.4 | 0.7 | 1.1 |
| total alkylated | 99.7 | 99.7 | 99.6 | 99.6 | 99.4 | 99.6 | 99.5 |
| DPE + DPP + PDPP sel, C-% | 0.03 | 0.07 | 0.07 | 0.08 | 0.08 | 0.07 | 0.06 |
| nPB/cumene | 133 | 90 | 300 | 126 | 929 | | 107 |

Spent Catalyst Carbon Levels

| | | | | | | |
|---|---|---|---|---|---|---|
| Top (C Content, %) | 5.0 | | 7.5 | 3.6 | | 5.02 |
| Mid (C Content, %) | 4.9 | | | | | |
| Bottom (C Content, %) | 3.8 | | 8.6 | 3.9 | | 4.78 |
| Whole Bed (C Content, %) | 4.6 | 2.5 | 8.1 | 3.8 | 2.7 | 4.9 |

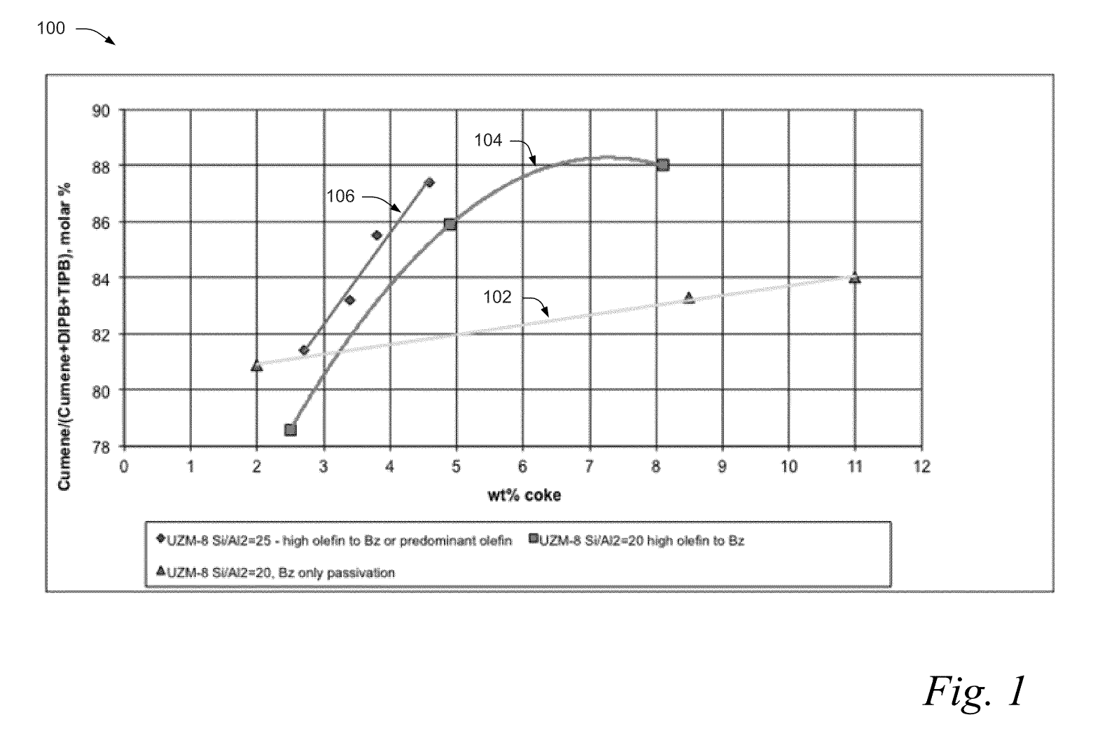

Referring to FIG. 1, a graph 100 of cumene selectivity for catalysts having varying amounts of carbonaceous material is depicted. The x-axis represents the weight percent of carbonaceous material as a result of Applicants' process. The y-axis represents the cumene selectivity (molar percentage of cumene over the sum total of cumene, DIPB and TIPB). Curve 102 represents the cumene selectivity trend for UZM-8 catalysts, with a Si/Al₂ molar ratio of about 20, treated with benzene to form a carbonaceous material on the surface thereof. Curve 104 represents the cumene selectivity trend for UZM-8 catalysts, with a Si/Al₂ molar ratio of about 20, treated with an olefin/benzene mixture having a high ratio of olefin to benzene. In certain embodiments, the olefin is propylene. Curve 106 represents the cumene selectivity trend for UZM-8 catalysts, with a Si/Al₂ molar ratio of about 25, treated with an olefin/benzene mixture having a very high ratio of olefin to benzene.

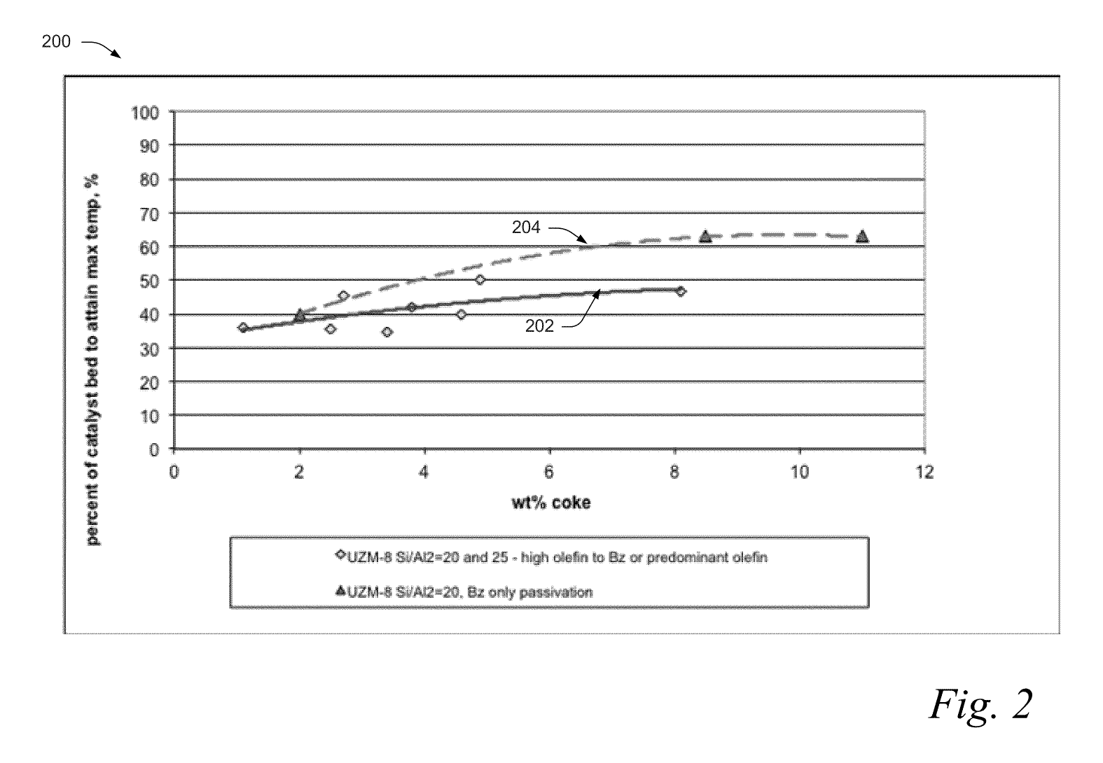

Referring to FIG. 2, a graph 200 of the portion of the catalyst bed necessary to attain maximum temperature (i.e., catalyst activity) for catalysts having various amounts of carbonaceous material is depicted. The x-axis represents the weight percent of carbonaceous material as a result of Applicants' process. The y-axis represents the percentage of the catalyst bed necessary to attain maximum temperature. Curve 202 represents the trend for UZM-8 catalysts, with a Si/Al₂ molar ratio of about 20 and about 25, treated with an olefin/benzene mixture having a high ratio of olefin to benzene. Curve 204 represents the trend for UZM-8 catalysts, with a Si/Al₂ molar ratio of about 20, treated with benzene only.

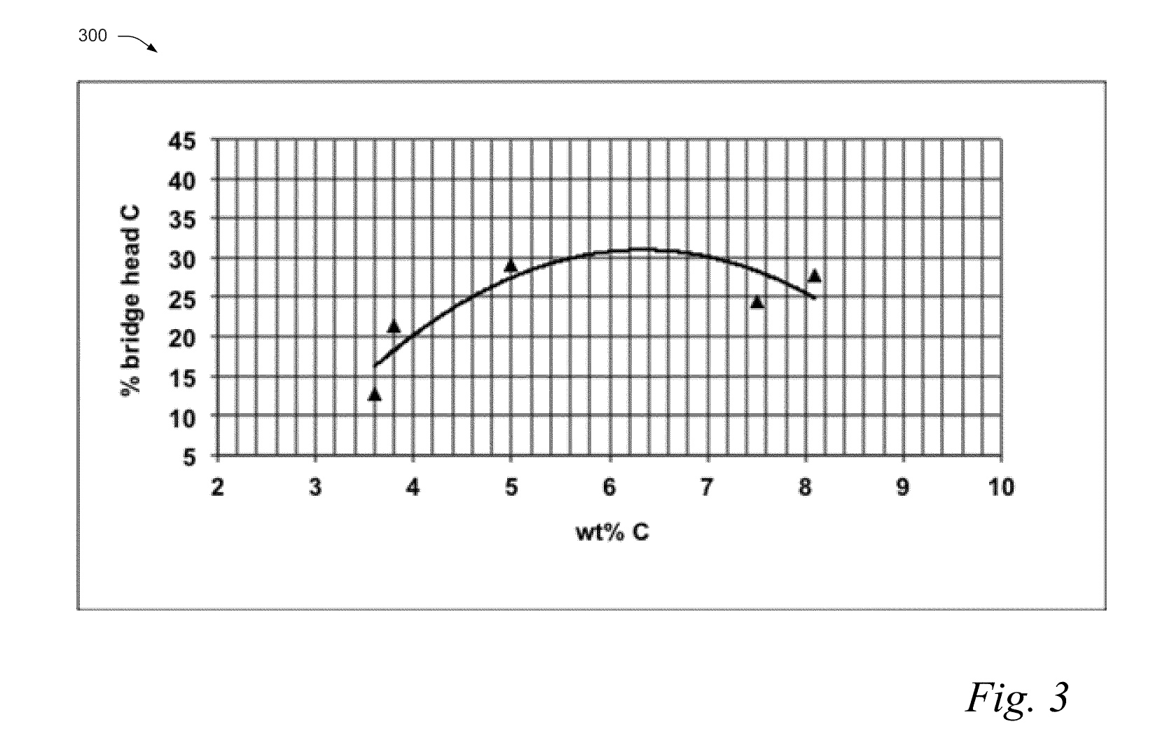

Referring to FIG. 3, a graph 300 of percent bridgehead carbon for catalysts having varying amounts of carbonaceous material is depicted. The x-axis represents the weight percent of carbonaceous material as a result of Applicants' process. The y-axis represents the percent of bridgehead carbon as a percentage of total carbonaceous material as determined by carbon-13 NMR analysis.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention. In other words, the present invention may be embodied in other specific forms without departing from its spirit or essential characteristics. The described implementations are to be considered in all respects only as illustrative and not restrictive. The scope of the invention should, therefore, be determined not with reference to the above description, but instead should be determined with reference to the pending claims along with their full scope or equivalents, and all changes which come within the meaning and range of equivalency of the claims are to be embraced within their full scope.

What is claimed is:

1. A composite, comprising:
a substrate, comprising a zeolite and an inorganic oxide; and
a carbonaceous material disposed on a surface of said substrate, wherein the carbonaceous material comprises greater than about 2.8 weight percent of said composite.

2. The composite of claim 1, wherein:
said zeolite comprises UZM-8;
said inorganic oxide comprises between about 20 weight percent and about 98 weight percent of said substrate; and
said inorganic oxide is selected from the group consisting of alumina, silica, magnesia, zirconia, and a combination thereof.

3. The composite of claim 1, wherein greater than about 10 percent of the carbonaceous material comprises bridgehead carbon.

4. The composite of claim 1, wherein said carbonaceous material is formed by the reaction of a hydrocarbon material in the vapor phase or partial vapor phase in the presence of said substrate, wherein said hydrocarbon material is selected from the group consisting of an olefin, an aromatic, and a combination thereof.

5. The composite of claim 4, wherein said zeolite has a Si/Al₂ molar ratio of about 19 to about 35.

6. The composite of claim 4, wherein:
said olefin is propylene;
said aromatic is benzene; and
and hydrocarbon material comprises propylene and benzene with a propylene/benzene ratio between about 0.001 to about 300.

7. The composite of claim 1, wherein the carbonaceous material comprises between about 3.5 weight percent to about 8 weight percent of said composite.

8. The composite of claim 1, prepared by the steps comprising:
providing a substrate comprising a zeolite and an inorganic oxide; and
depositing a carbonaceous material on a surface of said substrate, comprising exposing said substrate to a hydrocarbon material in the vapor phase or partial vapor phase, wherein said carbonaceous material comprises greater than about 2.8 weight percent of said composite catalyst.

9. The composite of claim 8, wherein:
said zeolite comprises UZM-8;
said inorganic oxide comprises between about 20 weight percent and about 98 weight percent of said substrate;
said inorganic oxide is selected from the group consisting of alumina, silica, magnesia, zirconia, and a combination thereof;
greater than about 10 percent of the carbonaceous material comprises bridgehead carbon;
said hydrocarbon material comprises propylene and benzene with a propylene/benzene ratio between about 0.001 to about 300; and
said exposing comprises a pressure between about 0.1 psia (0.7 kPa) to about 550 psia (3792 kPa) and a temperature between about 100° C. to 450° C. for between about 0.02 hours to about 144 hours.

10. A method of making a composite catalyst, comprising:
providing a substrate comprising a zeolite and an inorganic oxide; and
depositing a carbonaceous material on a surface of said substrate, comprising exposing said substrate to a hydrocarbon material in the vapor phase or partial vapor phase, wherein said carbonaceous material comprises greater than about 2.8 weight percent of said composite catalyst.

11. The method of claim 10, wherein;
said zeolite comprises UZM-8;
said inorganic oxide comprises between about 20 weight percent and about 98 weight percent of said substrate; and
said inorganic oxide is selected from the group consisting of alumina, silica, magnesia, zirconia, and a combination thereof.

12. The method of claim 10, wherein greater than about 10 percent of the carbonaceous material comprises bridgehead carbon.

13. The method of claim 10, wherein said hydrocarbon material is selected from the group consisting of an olefin, an aromatic, and a combination thereof.

14. The method of claim 11, wherein said zeolite has a Si/Al₂ molar ratio of about 19 to about 35.

15. The method of claim 13, wherein:
said olefin is propylene;
said aromatic is benzene; and
and hydrocarbon material comprises propylene and benzene with a propylene/benzene ratio between about 0.001 to about 300.

16. The method of claim 13, wherein the carbonaceous material comprises between about 3.5 weight percent to about 8 weight percent of said composite catalyst.

17. The method of claim 13, wherein said exposing comprises a pressure between about 0.1 psia (0.7 kPa) to about 550 psia (3792 kPa) and a temperature between about 100° C. to 450° C. for between about 0.02 hours to about 144 hours.

18. A method of making cumene, comprising:
providing a substrate comprising a zeolite and an inorganic oxide;
forming a composite catalyst by depositing a carbonaceous material on a surface of said substrate, comprising exposing said substrate to a hydrocarbon material in the vapor phase or partial vapor phase, wherein said carbonaceous material comprises greater than about 2.8 weight percent of said composite catalyst; and
forming cumene by exposing the composite catalyst to a stream comprising propylene and benzene.

19. The method of claim 18, wherein:
said zeolite comprises UZM-8;
said inorganic oxide comprises between about 20 weight percent and about 98 weight percent of said substrate; and
said inorganic oxide is selected from the group consisting of alumina, silica, magnesia, zirconia, and a combination thereof.

20. The method of claim 19, wherein:
greater than about 10 percent of the carbonaceous material comprises bridgehead carbon;
said hydrocarbon material is selected from the group consisting of an olefin, an aromatic, and a combination thereof;
said zeolite has a Si/Al₂ molar ratio of about 19 to about 35;
said carbonaceous material comprises between about 3.5 weight percent to about 8 weight percent of said composite catalyst; and
said exposing comprises a pressure between about 0.1 psia (0.7 kPa) to about 550 psia (3792 kPa) and a temperature between about 100° C. to 450° C. for between about 0.02 hours to about 144 hours.
